(19) United States
(12) Patent Application Publication
(10) Pub. No.: US 2015/0173692 A1
(43) Pub. Date: Jun. 25, 2015

(54) COMPUTED TOMOGRAPHY DEVICES, SYSTEMS, AND METHODS

(71) Applicant: University of Utah Research Foundation, Salt Lake City, UT (US)

(72) Inventor: Dominc Julius Heuscher, Salt Lake City, UT (US)

(21) Appl. No.: 14/579,528

(22) Filed: Dec. 22, 2014

Related U.S. Application Data
(60) Provisional application No. 61/934,449, filed on Jan. 31, 2014.
(60) Provisional application No. 61/919,114, filed on Dec. 20, 2013.

(51) Int. Cl. A61B 6/06; A61B 6/03; A61B 6/00

(52) U.S. Cl. CPC A61B 6/06; A61B 6/507; A61B 6/035; A61B 6/4028

(57) ABSTRACT

Computed tomography devices, systems, and associated methods are disclosed. The device can include a radiation source coupleable to a rotating gantry to emit radiation from an adjustable focal spot toward a volume of interest. The device can also include a dynamic collimator associated with the radiation source and located between the focal spot and the volume of interest. The dynamic collimator can define an aperture that is adjustable to permit radiation to pass through the volume of interest. The focal spot can be adjustable to maximize resolution within the volume of interest.

### RELATED APPLICATIONS

This application claims the benefit of U.S. Provisional Patent Application Nos. 61/919,114, filed Dec. 20, 2013, and 61/934,449 filed Jan. 31, 2014, each of which is incorporated herein by reference in its entirety.

### BACKGROUND

Computed tomography (CT) systems have included single-slice and multiple-slice detectors. CT systems with multiple-slice detectors are, in particular, able scan large volumes of interest. Some large volumes are imagined by helical CT scanning. In helical scanning, the subject moves axially relative to a radiation beam such that the beam traverses a helical path through the subject. Such scanning can be leveraged to quickly scan whole or large portions of organs.

### SUMMARY

A high radiation dose of conventional perfusion CT imaging can limit the clinical potential of CT. Due to high radiation doses, the CT scanning of kidneys, other abdominal organs, and the heart can be limited and have not been used routinely in various fields, including stroke assessment, oncology, and cardiac and kidney function. Cardiac and perfusion scans, in particular, have been limited in their clinical application because of the associated high x-ray dose to the patient during scans. Accordingly, improvements continue to be sought to avoid such drawbacks. The present technology can reduce the x-ray dose to the patient in situations where less than the full cross-section of the patient is being imaged. This is particularly applicable to clinical applications such as cardiac imaging and perfusion CT, which thus far have been limited in their use due to the high x-ray dose to the patient. In one aspect, the present technology can improve image quality or resolution, particularly for “off-center” volumes or regions of interest.

In one aspect, the present disclosure provides a computed tomography device. The device can include a radiation source coupleable to a rotating gantry to emit radiation from an adjustable focal spot toward a volume of interest. The device can also include a dynamic collimator associated with the radiation source and located between the focal spot and the volume of interest. The dynamic collimator can define an aperture that is adjustable to permit radiation to pass through the volume of interest. The focal spot can be adjustable to maximize resolution within the volume of interest.

In another aspect, the present disclosure provides a computed tomography system. The system can include a rotating gantry configured to rotate about a volume of interest. The system can also include a radiation source coupled to the gantry to emit radiation from an adjustable focal spot toward the volume of interest. The system can further include a dynamic collimator associated with the radiation source and located between the focal spot and the volume of interest. In addition, the system can include a radiation detector positioned opposite the radiation source and dynamic collimator to receive radiation that has passed through the volume of interest. The dynamic collimator can define an aperture that is adjustable to permit radiation to pass through the volume of interest. The focal spot can be adjustable to maximize resolution within the volume of interest.

In yet another aspect, the present disclosure provides a method of radiologic imaging. The method can comprise emitting radiation from a focal spot of a radiation source toward a volume of interest. The method can also comprise adjusting an aperture of a dynamic collimator located between the focal spot and the volume of interest to permit radiation to pass through the volume of interest. Additionally, the method can comprise adjusting the focal spot to maximize resolution within the volume of interest.

### BRIEF DESCRIPTION OF THE DRAWINGS

FIG. 1 is an illustration of a computed tomography system in accordance with an example of the present disclosure.

FIG. 2 illustrates limitation of radiation exposure to a region of interest at multiple positions of a dynamic collimator in accordance with an example of the present disclosure.

FIGS. 3A-3C are illustrations of a dynamic collimator in accordance with an example of the present disclosure.

FIGS. 4A-4C are illustrations of a dynamic collimator including shaping blades in accordance with another example of the present disclosure.

FIG. 5 is an illustration of a computed tomography device in accordance with an example of the present disclosure.

FIG. 6A illustrates mechanically rotating a focal spot of a radiation source.

FIG. 6B illustrates electronic rotation of a focal spot of a radiation source by applying an electrostatic field.

FIG. 6C illustrates electronic rotation of a focal spot of a radiation source by applying an electromagnetic field.

FIG. 7 is a schematic illustration of radiation source focal spot rotation coordinated with dynamic collimator aperture movement to scan an off-center volume or region of interest.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

In describing and claiming the present invention, the following terminology will be used.

The singular forms “a,” “an,” and “the” include plural referents unless the context clearly dictates otherwise. Thus, for example, reference to “a leaf” includes reference to one or more of such features and reference to “subjecting” refers to one or more such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

As used herein, the term “at least one of” is intended to be synonymous with “one or more of” For example, “at least one of A, B and C” explicitly includes only A, only B, only C, or combinations of each.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Although many features, aspects and embodiments are described herein or shown in the accompanying drawings in the context of CT, the disclosed technology can also be applied in other imaging systems and methods, other medical scenarios, or other image data acquisition or processing techniques.

During a helical CT scan, an x-ray source generates a cone (or wedge) beam of radiation that moves relative to the patient. Portions of the cone beam of radiation may not pass through the volume to be reconstructed. While this extra radiation may have little adverse effect on the clinical use of the reconstructed image, it can subject the patient to more radiation than is necessary. Primarily due to concerns about the magnitude of radiation dose delivered, perfusion CT imaging has not been used routinely in various fields, including stroke assessment, oncology, and cardiac and kidney function. Accordingly, various embodiments described herein relate to reducing or minimizing the magnitude of radiation dose delivered to an object being scanned or a selected area of the object being scanned.

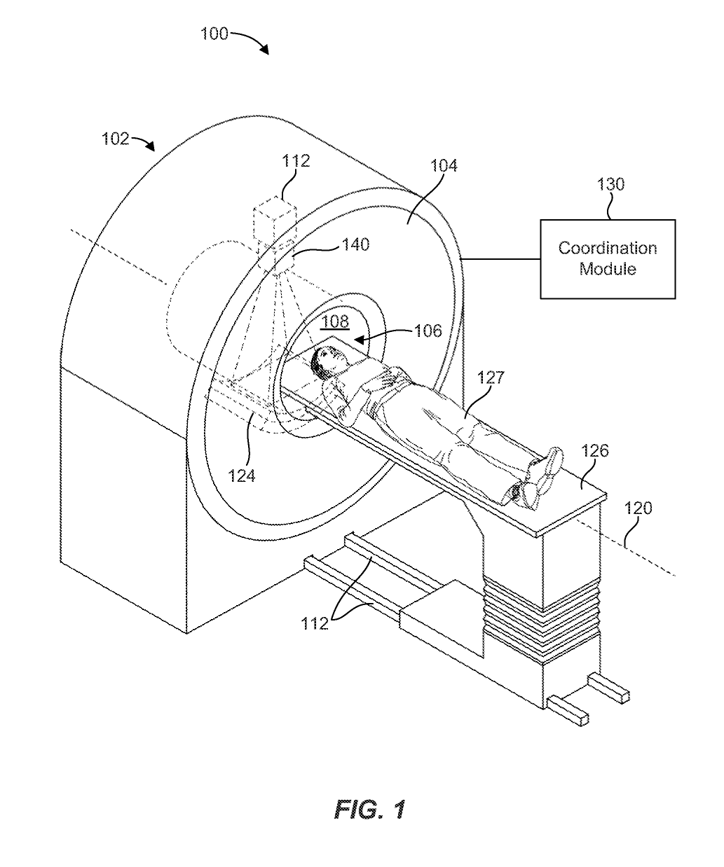

FIG. 1 illustrates an exemplifying CT imaging system 100 including a CT scanner 102 with a rotating gantry 104, a radiation source 112, a radiation detector 124, and a patient support 126 or couch. The gantry portion 104 can comprise a gantry opening 106 and can rotate about an examination region 108, which can include a volume of interest (VOI) to be scanned. The rotating gantry 104 can support the radiation source 112 and the detector 124.

The radiation source 112 can be an x-ray source, such as an x-ray tube, for example. The radiation source can emit a radiation beam. The radiation beam can be a cone beam, wedge beam, or other desirable beam shape. The beam can be collimated to have a generally conical geometry in some embodiments. In one aspect, discussed in more detail hereinafter, the radiation source 112 can emit radiation from an adjustable focal spot toward a volume of interest. The system 100 can include a dynamic collimator 140 associated with the radiation source 112 and located between the focal spot and the volume of interest. In one aspect, the dynamic collimator 140 can define an aperture that is adjustable to permit radiation to pass through the volume of interest. For example, the dynamic collimator 140 can include movable leaves and/or a movable grate that defines one or more apertures. In another aspect, the focal spot can be adjustable to maximize resolution within the volume of interest as described in more detail hereinafter. The system 100 can also include a coordination module 130 to coordinate adjustment of the focal spot with adjustment of the aperture. The function of the coordination module 130 is described in more detail below with regard to coordinated movement and adjustment of the radiation source and the dynamic collimator.

The detector 124 is sensitive to radiation (e.g., x-ray) emitted by the radiation source 112. In some embodiments, the detector 124 can be a detector array comprising multiple radiation detectors. The detector 124 can be disposed opposite the radiation source 112 on rotating gantry 104 to receive radiation that has passed through the volume of interest. In some embodiments, the detector 124 includes a multi-slice detector having a plurality of detector elements extending in the axial and transverse directions. Each detector element can detect radiation emitted by the radiation source 112 that traverses the examination region 108 and can generate corresponding output signals or projection data indicative of the detected radiation. Other detector configurations, such as those wherein stationary detectors surround the examination region, can also be used.

The motion of the radiation source and emission of radiation thereby are coordinated to scan a volume of interest such as anatomy, or a portion of anatomy, disposed within the examination region 108. The volume of interest can be enhanced with a contrast agent in some embodiments, such as described below, for example. In some embodiments, coordinated motion and radiation emission can be used for fly-by scanning, for example. In some embodiments, the radiation source and detector move in coordination with a contrast agent through the subject such that the VOI is scanned in coordination with the flow of the agent as it is traced through the VOI. In another embodiment, the axial advancement is coordinated with a motion of the subject to capture a desired motion state.

The support 126 can support a subject 127, such as a human patient for example, in which the VOI is defined within the examination region 108. A drive mechanism 116 can move the support 126 longitudinally along a z-axis 120 on tracks 128 while the CT scanner 102 is stationary and the radiation source 112 rotates in a fixed location about the z-axis. In some embodiments, however, the CT scanner 102 can be translated axially along the z-axis 120 while the support 126 is stationary. An operator of the system can define the VOI to encompass the whole subject or a portion thereof for scanning. In one embodiment, the CT scanner performs a helical scan of the VOI by rotating around the axis 120 during relative movement of the gantry and the support parallel to the axis.

In addition, to the coordination module 130, the system 100 can comprise various other computer hardware and/or software modules that facilitate operation and/or use of the system 100. For example, the system 100 can comprise data memory, a processor, a volume image memory, a user interface, and one or more controllers, such as a CT controller and a collimator controller. In some embodiments, a single hardware or software module can control multiple parts of the system, such as the radiation source 112 and one or more collimators 140, for example.

The projection data generated by the detector 124 can be stored to a data memory and reconstructed by a processor to generate a volumetric image representation therefrom. Thus, a plurality of projections can be used to form a complete scan from which a 2D and/or 3D image can be constructed using well known processing techniques. The reconstructed image data can be stored in a volume image memory and displayed to a user via a user interface. Although the data memory and the volume image memory can be separate, both can be stored within common data storage hardware. The image data can be processed to generate one or more images of the scanned region or volume of interest or a subset thereof.

A user interface can facilitate user interaction with the scanner 102 and can comprise various input and output devices. Software applications and modules can receive inputs from the user interface to configure and/or control operation of the scanner 102, and other elements of the system 100. For instance, the user can interact with the user interface to select scan protocols, and initiate, pause, and terminate scanning. The user interface can display images, facilitate manipulation of the data and images and measurement of various characteristics of the data and images, etc.

An optional physiological monitor can monitor cardiac, respiratory, or other motion of the VOI. For example, the monitor can include an electrocardiogram (ECG) or other device that monitors the electrical activity of the heart. This information can be used to trigger one or more scans or to synchronize scanning with the heart electrical activity to reduce or eliminate adverse effects of heart motion on imaging. An optional injector or the like can be used to introduce agents, such as contrast for example, into the subject. Introduction of the agent can also be used to trigger one or more scans.

A CT controller can control rotational and axial movement of the radiation source 112 and the detector 124 relative to the support 126. The CT scanner 102 and the CT controller can be coupled to a collimator controller that controls the dynamic collimator 140, which is positioned between the radiation source 112 and the examination region 108. Although the CT controller and the collimator controller can be separate units, the CT scanner 102 and one or more collimators 140 can be controlled by the same hardware and software modules.

The collimator controller can control movement, and opening and closing, of an aperture or radiation delivery window of the collimator 140. In some embodiments, the collimator controller can independently control movement of individual leaves of the collimator. The collimator controller can be a software module configured to move leaves of the collimator 140 that define an aperture to allow passage of radiation toward a region of interest while blocking radiation to portions of a subject outside the region of interest.

In some embodiments, the collimator controller can cause the collimator 140 to function as a shutter to block radiation between scans and to open, close, and translate as the rotatable gantry 104 (and, accordingly, the source unit 112 and detector 124 coupled thereto) moves around the VOI during a scan. In some embodiments, the collimator controller can include one or more electro-mechanical servo motors. In some embodiments, the collimator controller can include an electronic controller.

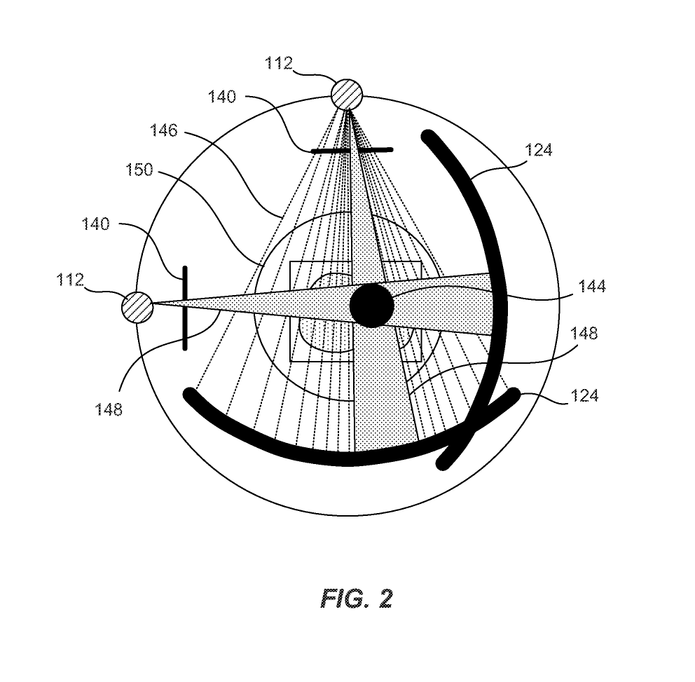

Repeated large area circular scans and helical scans can be used to perform perfusion CT. By opening, closing, and/or translating the collimator 140, radiation can be delivered primarily only along paths that intersect the VOI, thereby reducing the x-ray dose. In the case of helical scans, a dynamic axial collimator can be used to limit the x-ray exposure, axially, at either end or both ends of the helical scan. For example, in some embodiments, an axial collimator can be gradually opened at the leading end of the VOI and closed at the trailing end of the VOI. A dynamic transverse collimator positioned in a plane transverse to a gantry rotation axis and in front of the radiation source can limit the x-ray exposure to a region of interest (ROI) within the VOI in a field of view of the radiation source, as illustrated in FIG. 2. FIG. 2 is a schematic illustration of an imaging system showing two positions (at 0 and 90 degrees) of a radiation source 112, a detector 124, and a dynamic collimator 140 relative to a subject 142 for an off-center ROI 144 surrounding the subject's heart. In one aspect, a VOI can correspond to the heart although other tissues and organs can certainly be targeted as a VOI. Transverse and axial collimators can together limit the x-ray exposure to primarily only the VOI for the heart, for example. An full field radiation beam 146 represents a typical non-collimated beam. The dynamic collimator 140 can narrow the radiation to a dynamically changing reduced radiation beam 148. Without collimated, an radiation exposed volume would be defined by the full scan field circle 150. With dynamic collimation as described herein, the scan field circle can be limited to a specific ROI 144. In another aspect, a VOI can include multiple ROIs, each of which can surround the subject's heart as defined by the dynamic collimator for the radiation source at a given position.

As illustrated in FIGS. 1 and 2, the dynamic collimator 140 can be positioned between the radiation source 112 and the VOI or ROI 144 and can be moveable with the radiation source 112. The radiation detector 124 can be positioned opposite the radiation source and the collimator to receive radiation that has passed through the VOI or ROI. The radiation source can begin image acquisition of the VOI or ROI at an initiation position and terminate image acquisition of the VOI or ROI at a termination position. In one aspect, the dynamic collimator can open at an x-ray initiation position to limit x-rays passing through the collimator to x-rays that will pass through the VOI or ROI. Examples of utilizing dynamic collimators to limit x-ray exposure can be found in U.S. Pat. No. 8,213,568, and U.S. Patent Application Publication Nos. 2013/0343513 and 2013/0343514, each of which is incorporated herein by reference in its entirety. In another aspect, the dynamic collimator can open and/or move in coordination or synchronization with movement or rotation of the focal spot to enable a resulting x-ray cone beam to pass through the aperture to scan the VOI or ROI. Rotation of the focal spot can improve image resolution for off-center volumes or regions of interest.

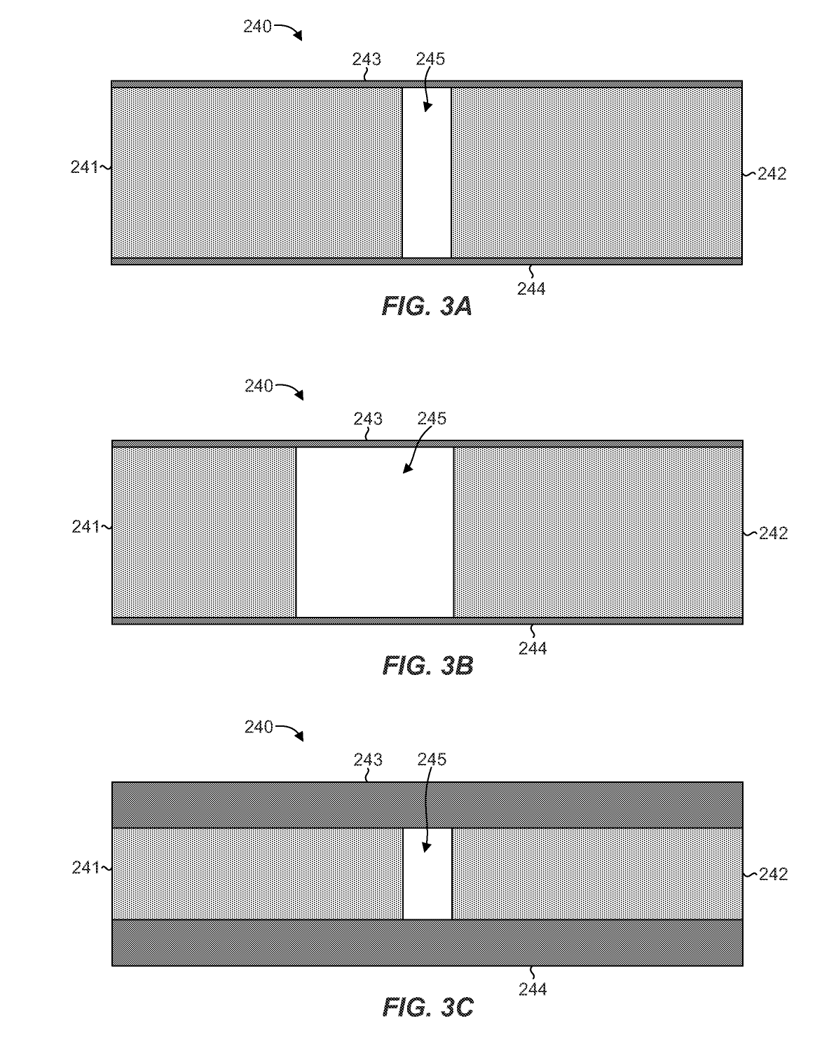

As illustrated in FIGS. 3A-3C, a dynamic collimator 240 can comprise a first leaf 241 and a second leaf 242 bounding first and second opposing sides of an aperture 245 or radiation delivery window, respectively. The first leaf and the second leaf can be movable to define and adjust a size and/or a position of the aperture 245 (see, e.g., FIGS. 3B and 3C) relative to a radiation source in a direction non-parallel to a longitudinal z-axis or axis of rotation of the radiation source (i.e., gantry rotation). The first leaf and the second leaf can be independently movable relative to one another and the radiation source in a direction non-parallel to the axis. In some embodiments, the first leaf and the second leaf can be independently movable relative to the radiation source in a direction tangential to a circle centered on the z-axis and defining a plane that is not parallel to the z-axis. In some embodiments, the leaves can be movable along guide rails. In one aspect, the leaves can move in linear or arcuate paths.

In some embodiments, the collimator can comprise a third leaf 243 and a fourth leaf 244 bounding the third and fourth opposing sides of the aperture 245, respectively, and can be movable to define and adjust a size and/or a position of the aperture. Each of the first and second sides of the aperture 245 can be substantially orthogonal to the third and fourth opposing sides of the aperture 245. The third leaf and the fourth leaf can be arranged generally along a line that is parallel to the axis of gantry or radiation source rotation. The aperture 245 can be interposed between the third and fourth leaves such that radiation is transmitted between the third and fourth leaves in a direction generally perpendicular to the axis of rotation. The third leaf and the fourth leaf can be independently movable relative to one another and the radiation source with a direction of motion being generally parallel to the axis of rotation. In some embodiments, the third and fourth leaves 234, 244 can be movable independently of the first and second leaves 241, 242.

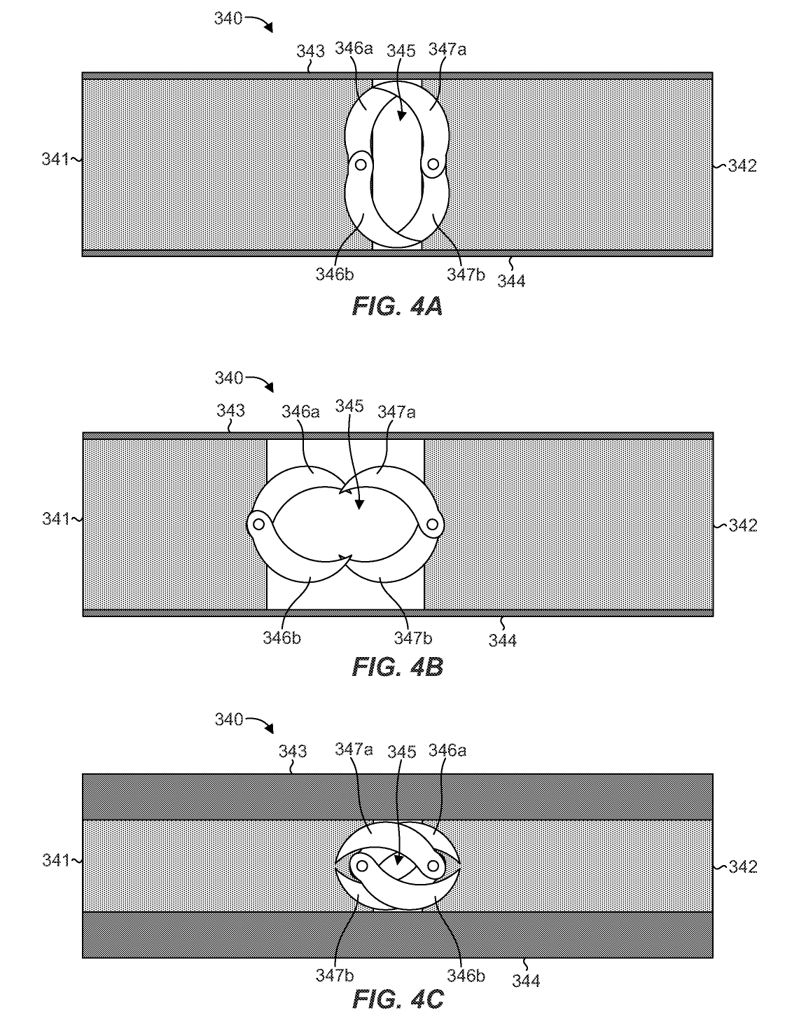

FIGS. 4A-4C illustrate a dynamic collimator 340 in accordance with another example of the present disclosure. As with the dynamic collimator 240 of FIGS. 3A-3C, the dynamic collimator 340 can include a first leaf 341 and a second leaf 342, respectively, bounding first and second opposing sides of an aperture 345 or radiation delivery window and a third leaf 343 and fourth leaf 344, respectively, bounding the third and fourth opposing sides of the aperture 345.

In this case, the dynamic collimator 340 can also include rotatable shaping or collimator blades 346a, 346b, 347a, 347b to adjust a size of the aperture 345. In one aspect, the shaping blades can be arranged in pairs and rotatably coupled to one or more leaves of the collimator 340. For example, a first pair of shaping blades 346a, 346b can be coupled to the first leaf 341 and a second pair of shaping blades 347a, 347b can be coupled to the second leaf 342 to adjust the size of the aperture 345. The figures show that the shaping blades can be attached to the ends of leaves of the dynamic collimator 340, although such can alternatively be attached to a static collimator. A radiation dose to an object can be reduced during a CT scan by dynamically adjusting the position of the shaping blades attached to the dynamic collimator to control or limit an x-ray cone beam that passes through a VOI and to block a portion of the x-ray cone beam that does not pass through the VOI. Thus, the shaping blades can be rotated “open” or “closed” depending on the shape or profile of the volume being scanned to match the shape as closely as possible. As the radiation source rotates about an axis, the dynamic collimator can be adjusted to adjust the width and/or location of the radiation beam so that radiation is primarily allowed to pass through the volume of interest and to block some or all of the rays of radiation that will not intersect the volume of interest. Thus, as the radiation source rotates, the leaves 341, 342 can independently adjust for an off-center position and projected width of the VOI, while the shaping blades adjust for the projected length and shape of the object. One advantage of attaching the shaping blades to the leaves of a dynamic collimator is that the VOI may be exposed only within its projected outline, with a minimal increase in the overall complexity of the dynamic collimator.

In one aspect, the shaping blades can allow the dynamic collimator to accommodate various 3D shapes when performing cone-beam scans. In general, the shaping blades reduce radiation dosage in proportion to the size of the cone-beam. Thus, for a large cone-beam, the dose reduction due to the shaping blades is greater than for a small cone-beam. In some cases, dose reduction due to shaping blades can exceed 40% for cone-beams that encompass the entire volume or region of interest.

The dynamic shaping blades can target three dimensional (3D) volumes when performing cone-beam scans. In one aspect, an angle or rotational position of the shaping blades and/or a width of an aperture of a dynamic collimator can vary with the size and shape of the profile of the 3D volume of interest as the radiation source rotates, thus adjusting the size of the cone beam and reducing or minimizing the radiation dose. One advantage of shaping blades can be to provide additional radiation dose savings or reduce radiation dosage over a typical dynamic leaf collimator. For example, when scanning an entire VOI (e.g., an ellipsoid), depending on its orientation, the integrated shaping blades can further reduce the radiation dose for a CT scan by over 21 percent compared to a typical dynamic leaf collimator.

In one aspect, transverse and axial collimators can be driven by the same motor or different motors. Similarly, transverse and axial collimators can be controlled by the same hardware or software modules. In some embodiments, transverse and axial collimators can be integrated into a single unit.

The VOI can be defined, for example, by a previously-acquired very low dose scan of the same region or two orthogonal localizer scans could be used. An operator can specify an axial extent of the VOI and the size, shape, and location of each ROI along the axial direction. In some embodiments, an outline of the entire VOI can be drawn from two orthogonal views, e.g. sagittal and coronal views, with the images zoomed according to the largest ROI in the sequence. The truncated region of each reconstructed image can be displayed with a dark background.

Axial collimator leaves can be opened and closed based on the axial extent of the VOI. If the VOI is modeled using elliptical cross-sections, transverse collimator leaves can move smoothly as they closely follow the outline of the VOI. In the case of a large cone-beam, the beam already encompasses a large portion of the VOI, therefore there can be less narrowing of the VOI profile at the ends of the scan.

As in the case of an axial collimator, the positions dynamic transverse collimator leaves can be based on the support position. However, in the case of the dynamic transverse collimator, as the support moves in the axial direction, the rotation angle can be used to determine where the current ROI is situated with respect to the source. Given both the support position and rotation angle, the leaves can continuously follow the outline of the overall VOI. For example, in the case of the cardiac scan, the collimator leaves can follow ROIs located along the cardiac volume based on the support location of the ROI as well as the rotation angle of the radiation source. The ROI along the cardiac volume can have both a non-circular (e.g., elliptical) shape and a location away from a scan center.

In some embodiments, for a sequence of axial or circular scans, one ROI can be determined for each scan in the sequence. For each scan, the rotation angle can be used alone to determine the motion of the collimator leaves, adjusting for an off-center and/or non-circular ROI.

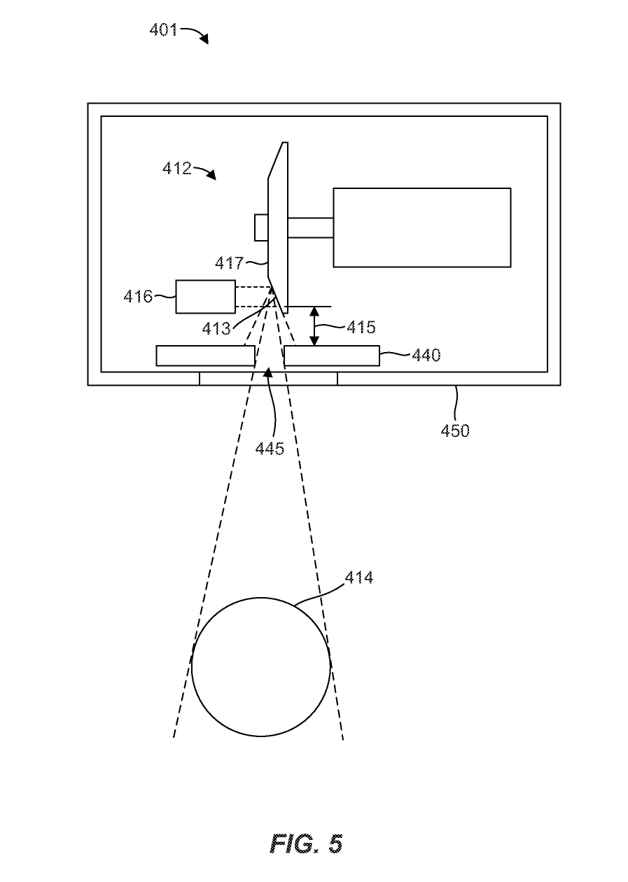

FIG. 5 illustrates a computed tomography device 401 in accordance with an example of the present disclosure. The device 401 can include a radiation source 412 coupleable to a rotating gantry to emit radiation from an adjustable focal spot 413 toward a volume or region of interest 414. The device 401 can also include a dynamic collimator 440 associated with the radiation source 412 and located between the focal spot 413 and the volume or region of interest 414. The dynamic collimator 440 can define an aperture 445 or collimator opening that is adjustable to permit radiation to pass through the volume or region of interest 414.

In one aspect, a distance 415 between the focal spot 413 and the aperture 445 is less than or equal to about 12 centimeters. In a particular aspect, the distance 415 between the focal spot 413 and the aperture 445 is from about 10 centimeters to about 12 centimeters. In another particular aspect, the distance 415 between the focal spot 413 and the aperture 445 is from about 8 centimeters to about 10 centimeters. Locating the dynamic collimator 440 relatively close to the focal spot 413 can reduce or minimize the velocity requirements of the dynamic collimator 440. In other words, by reducing the distance between the collimator 440 and the focal spot 413, the distances traveled by the collimator leaves are also reduced, thus reducing the speed at which the leaves must move or translate during the course of a scan, resulting in less collimator motion and slower velocities. In addition, such close proximity of the collimator 440 and the focal spot 413 can facilitate disposing the radiation source 412 and the dynamic collimator 440 within a common housing 450 to which these components can be affixed or coupled. Thus, the radiation source 412 and the dynamic collimator 440 can be contained within the same unit and coupled to a rotating gantry via the housing 450, which can improve performance and simplify the design of the collimator.

In one aspect, the focal spot 413 can be adjustable (e.g., in orientation, shape, and/or size) to influence (i.e., maximize) resolution and image quality of a radiographic image within the volume or region of interest 414 by providing isotropic resolution within the volume or region. For example, rotating the focal spot can improve resolution within an off-center volume or region of interest by more than a factor of two. As shown in FIG. 5, the radiation source 412 can include a cathode 416 to emit electrons and an anode 417 to receive the electrons at the focal spot 413, from which radiation is emitted toward the volume or region of interest 414. The orientation, shape, and/or size of the focal spot 413 can be altered or adjusted by manipulating an angle and/or length of a cathode filament of the cathode 416.

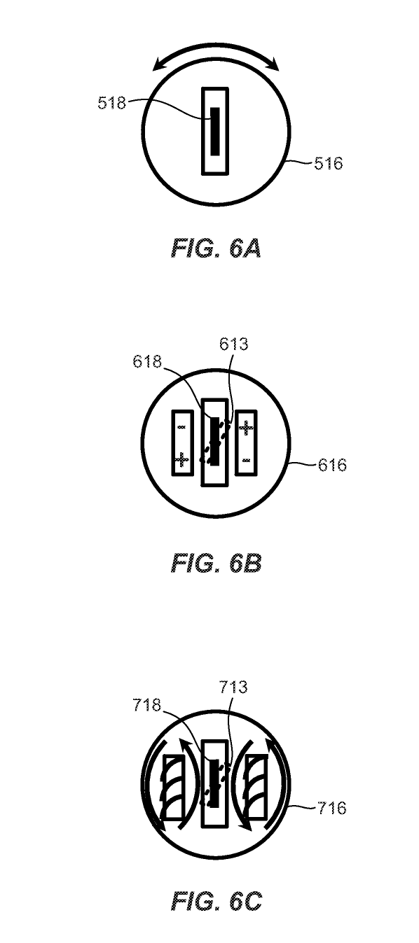

FIGS. 6A-6C illustrate several examples of rotating a focal spot. In FIG. 6A, an angle or orientation of a focal spot can be adjusted by mechanically rotating a cathode 516 and/or a cathode filament 517, such as in a clockwise or counterclockwise direction. FIG. 6B shows that an angle or orientation of a focal spot 613 can be adjusted by electronic angulation by applying an electrostatic field in front of a cathode 616 and/or a cathode filament 618 to twist or rotate electrons emitted from the cathode 616. Similarly, FIG. 6C shows that an angle or orientation of a focal spot 713 can be adjusted by electronic angulation by applying an electromagnetic field in front of a cathode 716 and/or a cathode filament 718 to twist or rotate electrons emitted from the cathode 716. Such electrostatic and electromagnetic fields can be generated and controlled by any suitable device or mechanism known in the art.

The focal spot can be varied or adjusted in orientation, shape, and/or size to direct radiation at a target volume or region of interest, such as to encompass a volume or region of interest that is eccentrically located relative to an axis of rotation of a rotating gantry. In one aspect, adjustment of a size of the focal spot can be coordinated with adjustment of a size of the aperture of the dynamic collimator. In another aspect, adjustment of an orientation of the focal spot can be coordinated with adjustment of a position of the aperture of the dynamic collimator.

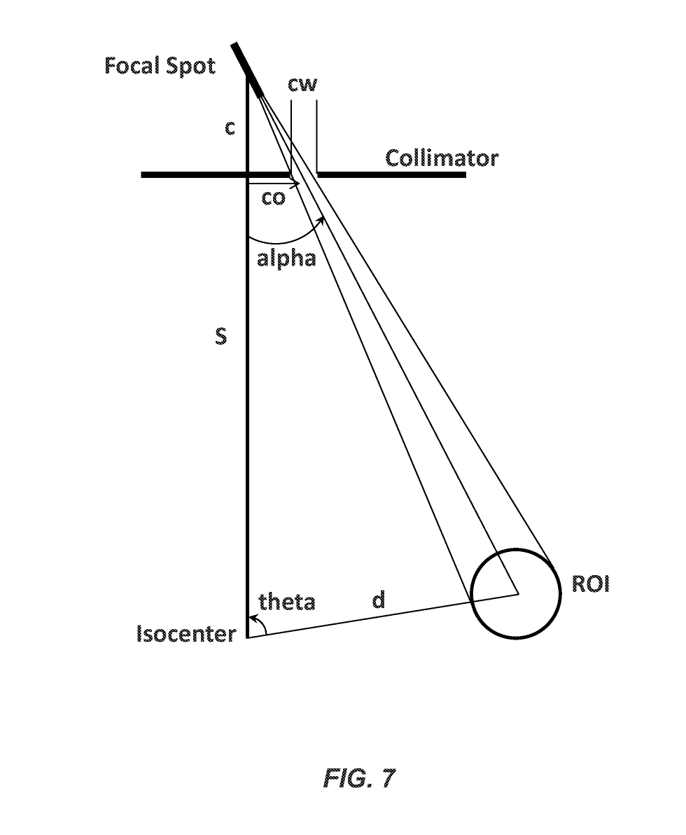

For example, the movement or rotation of the focal spot can be synchronized or coordinated with the movement of the aperture or operation of the dynamic collimator, such as for an off-center or eccentrically located volume or region of interest schematically illustrated in FIG. 7, by using the following equations:

$$alpha = atan ( \frac{d sin ( theta )}{s - d cos ( theta )} ) , \\ co = c tan ( alpha ) + r \frac{\sqrt{{c}^{2} + {o}^{2}}}{2 l} ( \frac{1}{cos ( alpha )} - cos ( alpha ) ) , and$$

$$cw = r \frac{\sqrt{{c}^{2} + {o}^{2}}}{2 l} ( \frac{1}{cos ( alpha )} + cos ( alpha ) )$$

where alpha is an angle of the radiation source focal spot, co is a collimator offset, cw is a width of collimator opening, I²=(S-d cos(theta))²+(d sin(theta))², o=C tan(alpha), theta is a rotation angle of the radiation source focal spot relative to the ROI, c equals the of collimator distance from the radiation source focal spot, d equals the distance of the ROI center from a center of the scan area of the object, and S equals a distance from the radiation source focal spot to an isocenter. In one embodiment, for each angle theta, the collimator offset, co, collimator width, cw, and radiation source focal spot angle alpha can be updated. Thus, the focal spot can be rotated to maximize the resolution within off-center volumes or regions of interest defined by the dynamic collimator. Such rotation can improve the image quality within these regions by providing isotropic resolution within the region.

In one aspect, the dynamic collimator and the radiation source can both be controlled by a signal that defines the movement of the collimator leaves and/or blades as well as the rotation of the focal spot. In one embodiment, a control circuit, which can be included in a coordination module, can generate a signal to synchronize the collimator motion and an angulation of the focal spot. A model of a control signal can be generated by the coordination module and used to translate the control signal using the above geometric equations to a signal that generates an angulation, such as an electromagnetic control or electrostatic control, and a required linear motion of the collimator, such as a linear solenoid control, a linear motor, or a stepper motor. The integration of focal spot motion with collimator motion can simplify subsystem design, reduce cost, and allow the dynamic collimator aperture to be positioned closer to the focal spot, as discussed above.

The physical integration or combination of a dynamic collimator with a radiation source into a common unit can simplify the accurate synchronizing of the rotation of the focal spot with the sizing and/or positioning of the collimator aperture. For example, coordination or synchronization of the dynamic collimator with axial movement of the radiation source can reduce x-rays passing through the collimator as the collimator reaches the x-ray termination position. Additionally, when reconstructing the images of the ROI, the CT scanner can incorporate penumbra information into the ROI reconstruction. By accurately knowing the position of the dynamic collimator aperture, knowledge of the penumbra can be incorporated into the ROI reconstruction, thus improving image quality and reducing loss in dose efficiency.

In one aspect, a predefined VOI can be used to control the dynamic collimator and focal spot of the radiation source. In one embodiment, the images from the dynamically-collimated scans can be reconstructed by obtaining orthogonal x-ray projections and using profiles of the orthogonal x-ray projections of the desired object to generate the predefined VOI approximated by elliptical cross-sections. To avoid inaccurate reconstructions due to the missing projection data, data can be collected in the truncated region during the CT scan of the VOI either using a grating collimator or by strongly or highly attenuating x-rays in the truncated regions.

In another aspect, images can be reconstructed by using a very low radiation dose CT scan with no transverse collimation covering the extent of the anatomy to generate the predefined VOI, optionally from a previous scan. Non-truncated projections from the very low radiation dose scan can be used to complete projections of the VOI scan. To adjust for slight registration errors between the very low dose scan and the VOI scan, the image data within the VOI of the two reconstructions can be correlated to provide registration parameters then used to recalculate the non-truncated projections. These new projection data can then be used to complete the VOI projections and produce a more accurate set of images from the VOI scan. Within the VOI the high frequency edges will correlate, and low frequency error outside the VOI can be attributed to registration errors.

Using a radiation source and a dynamic collimator physically incorporated into a common unit can enable modeling of the penumbra with a collimator close to the x-ray source. When the penumbra is modeled with a collimator close to the x-ray source, missing projection data can be seamlessly combined with the VOI scan data. Additionally, all the penumbra data can be utilized to avoid an increase in effective radiation dose. In one embodiment, air scans can be performed at different collimator positions to assure the model is accurate.

The principles disclosed herein can enable a dynamic collimator and a radiation source to target an anatomy of interest and lower the x-ray dose to a patient in all situations where less than the full cross-section of the patient is to be imaged. Such dose reduction capabilities can increase the use of CT scanning in clinical applications, such as cardiac imaging and perfusion CT, which has been limited in use due to the high x-ray dose to the patient. In addition to reducing x-ray dose, the dynamic collimator and radiation source with a rotating focal spot can improve image quality and resolution in off-center target regions of the anatomy. The principles disclosed herein can also simplify the implementation of a dynamic collimator and provide shaping blades that allow the collimator to better match the shape of the VOI to further reduce the x-ray dose.

In accordance with one embodiment of the present invention, a method of radiologic imaging is disclosed. The method can comprise emitting radiation from a focal spot of a radiation source toward a volume of interest. The method can also comprise adjusting an aperture of a dynamic collimator located between the focal spot and the volume of interest to permit radiation to pass through the volume of interest. Additionally, the method can comprise adjusting the focal spot to maximize resolution within the volume of interest. It is noted that no specific order is required in this method, though generally in one embodiment, these method steps can be carried out sequentially.

In one aspect, the method can further comprise coordinating adjustment of an orientation of the focal spot with adjustment of a position of the aperture. In another aspect, the method can further comprise coordinating adjustment of a size of the focal spot with adjustment of a size of the aperture.

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A computed tomography device, comprising:
a radiation source coupleable to a rotating gantry to emit radiation from an adjustable focal spot toward a volume of interest; and
a dynamic collimator associated with the radiation source and located between the focal spot and the volume of interest,
wherein the dynamic collimator defines an aperture that is adjustable to permit radiation to pass through the volume of interest, and
wherein the focal spot is adjustable to maximize resolution within the volume of interest.

2. The computed tomography device of claim 1, wherein the focal spot is adjustable in at least one of orientation, shape, and size.

3. The computed tomography device of claim 1, wherein the radiation source comprises a cathode to emit electrons and an anode to receive the electrons at the focal spot and emit radiation therefrom.

4. The computed tomography device of claim 3, wherein an orientation of the focal spot is adjustable by mechanically rotating the cathode.

5. The computed tomography device of claim 3, wherein an orientation of the focal spot is adjustable by applying an electrostatic field to the electrons emitted from the cathode.

6. The computed tomography device of claim 3, wherein an orientation of the focal spot is adjustable by applying an electromagnetic field to the electrons emitted from the cathode.

7. The computed tomography device of claim 1, wherein the dynamic collimator comprises at least two movable leaves to define at least one of a size and a position of the aperture.

8. The computed tomography device of claim 7, wherein the dynamic collimator further comprises a first pair of shaping blades coupled to a first leaf and a second pair of shaping blades coupled to a second leaf, wherein the first and second pairs of leaves are rotatable to adjust the size of the aperture.

9. The computed tomography device of claim 1, wherein the dynamic collimator comprises rotatable shaping blades to adjust a size of the aperture.

10. The computed tomography device of claim 1, wherein adjustment of the focal spot is coordinated with adjustment of the aperture.

11. The computed tomography device of claim 1, further comprising a housing, wherein the radiation source and the dynamic collimator are disposed within the housing, and wherein the radiation source is coupleable to the rotating gantry via the housing.

12. The computed tomography device of claim 1, wherein a distance between the focal spot and the aperture is less than or equal to about 12 centimeters.

13. The computed tomography device of claim 12, wherein the distance between the focal spot and the aperture is from about 10 centimeters to about 12 centimeters.

14. A computed tomography system, comprising:
a rotating gantry configured to rotate about a volume of interest;
a radiation source coupled to the gantry to emit radiation from an adjustable focal spot toward the volume of interest;
a dynamic collimator associated with the radiation source and located between the focal spot and the volume of interest; and
a radiation detector positioned opposite the radiation source and dynamic collimator to receive radiation that has passed through the volume of interest,
wherein the dynamic collimator defines an aperture that is adjustable to permit radiation to pass through the volume of interest, and
wherein the focal spot is adjustable to maximize resolution within the volume of interest.

15. The computed tomography system of claim 14, further comprising a coordination module to coordinate adjustment of the focal spot with adjustment of the aperture.

16. The computed tomography system of claim 15, wherein adjustment of an orientation of the focal spot is coordinated with adjustment of a position of the aperture.

17. The computed tomography system of claim 15, wherein adjustment of a size of the focal spot is coordinated with adjustment of a size of the aperture.

18. The computed tomography system of claim 14, wherein the volume of interest is eccentrically located relative to an axis of rotation of the rotating gantry.

19. The computed tomography system of claim 14, further comprising a housing, wherein the radiation source and the dynamic collimator are disposed within the housing, and wherein the radiation source is coupled to the rotating gantry via the housing.

20. A method of radiologic imaging, comprising:
emitting radiation from a focal spot of a radiation source toward a volume of interest;
adjusting an aperture of a dynamic collimator located between the focal spot and the volume of interest to permit radiation to pass through the volume of interest; and
adjusting the focal spot to maximize resolution within the volume of interest.

21. The method of claim 20, further comprising coordinating adjustment of an orientation of the focal spot with adjustment of a position of the aperture.

22. The method of claim 20, further comprising coordinating adjustment of a size of the focal spot with adjustment of a size of the aperture.
